(19) United States
(12) Patent Application Publication
(10) Pub. No.: US 2024/0245666 A1
(43) Pub. Date: Jul. 25, 2024

(54) DOSING REGIMENS

(71) Applicant: Novartis AG, Basel (CH)

(72) Inventors: Stephanie Kay DODD, Ayer, MA (US); Florence HOURCADE-POTELLERET, Landser (FR); Felix HUTH, Muttenz (CH); Michelle QUINLAN, Morris Plains, NJ (US); Julia ZACK, Jersey City, NJ (US)

(21) Appl. No.: 18/559,859

(22) PCT Filed: May 10, 2022
(86) PCT No.: PCT/IB2022/054321

§ 371 Date: Nov. 9, 2023

Related U.S. Application Data
(60) Provisional application No. 63/187,023, filed on May 11, 2021.

(51) Int. Cl. A61K 31/4439; A61K 31/506; A61K 45/06

(52) U.S. Cl. CPC A61K 31/4439; A61K 31/506; A61K 45/06

(57) ABSTRACT

The present disclosure relates to dosing regimens and combinations comprising N-[4-(Chlorodifluoromethoxy)phenyl]-6-[(3R)-3-hydroxypyrrolidin-1-yl]-5-(1H-pyrazol-5-yl)pyridine-3-carboxamide or a pharmaceutically acceptable salt thereof, and their use for the treatment of breakpoint cluster region-abelson protein (BCR-ABL) mediated diseases or disorders.

### TECHNICAL FIELD

The present disclosure relates to dosing regimens and combinations comprising N-[4-(Chlorodifluoromethoxy)phenyl]-6-[(3R)-3-hydroxypyrrolidin-1-yl]-5-(1H-pyrazol-5-yl)pyridine-3-carboxamide or a pharmaceutically acceptable salt thereof, and their use for the treatment of breakpoint cluster region-abelson protein (BCR-ABL) mediated diseases or disorders.

### BACKGROUND

The tyrosine kinase activity of the ABL1 protein is normally tightly regulated, with the N-terminal cap region of the SH3 domain playing an important role. One regulatory mechanism involves the N-terminal cap glycine-2 residue being myristoylated and then interacting with a myristate binding site within the SH1 catalytic domain. A hallmark of chronic myeloid leukemia (CML) is the Philadelphia chromosome (Ph), formed by the t(9,22) reciprocal chromosome translocation in a haematopoietic stem cell. This chromosome carries the BCR-ABL1 oncogene which encodes the chimeric BCR-ABL1 protein, that lacks the N-terminal cap and has a constitutively active tyrosine kinase domain.

Although drugs that inhibit the tyrosine kinase activity of BCR-ABL1 via an ATP-competitive mechanism, such as Gleevec®/Glivec® (imatinib), Tasigna® (nilotinib) and Sprycel® (dasatinib), are effective in the treatment of CML, some patients relapse due to the emergence of drug-resistant clones, in which mutations in the SH1 domain compromise inhibitor binding. Although Tasigna® and Sprycel® maintain efficacy towards many Gleevec-resistant mutant forms of BCR-ABL1, the mutation in which the threonine-315 residue is replaced by an isoleucine (T315I) remains insensitive to all three drugs and can result in CML patients developing resistance to therapy. Therefore, inhibiting BCR-ABL1 mutations, such as T315I, remains an unmet medical need. In addition to CML, BCR-ABL1 fusion proteins are causative in a percentage of acute lymphocytic leukemias, and drugs targeting ABL kinase activity also have utility in this indication.

Agents targeting the myristoyl binding site (so-called allosteric inhibitors) have potential for the treatment of BCR-ABL1 disorders (Zhang et. al., Targeting BCR-ABL by combining allosteric with ATP-binding-site inhibitors. Nature 2010; 463:501-6). To prevent the emergence of drug resistance from ATP inhibitor and/or allosteric inhibitor use, a combination treatment using both types of inhibitor can be developed for the treatment of BCR-ABL1 related diseases or disorders. In particular, the need exists for small molecules, or combinations thereof, that inhibit the activity of BCR-ABL1 and BCR-ABL1 mutations via the ATP binding site, the myristoyl binding site or a combination of both sites.

Herein is provided a BCR-ABL inhibitor with activity at a site distinct from currently available ATP-site second- and third-generation TKIs, which may present an alternative mechanism of inhibition and, if used in combination, may prevent the development of resistance due to the acquisition of point mutation(s) being acquired in one of the binding sites, and thus address the unmet medical need, including treating an BCR-ABL-mediated disease or disorder that include CML, ALL, and AML.

### SUMMARY OF THE DISCLOSURE

Described herein are methods of treating a subject using a BCR-ABL inhibitor, in particular Compound I, for use in treating a BCR-ABL-mediated disease or disorder. Described herein are also methods of treating a BCR-ABL-mediated disease or disorder by administering to a subject in need thereof a therapeutically effective amount of a BCR-ABL inhibitor, in particular Compound I, administered without food.

The compound N-[4-(Chlorodifluoromethoxy)phenyl]-6-[(3R)-3-hydroxypyrrolidin-1-yl]-5-(1H-pyrazol-5-yl)pyridine-3-carboxamide of the formula

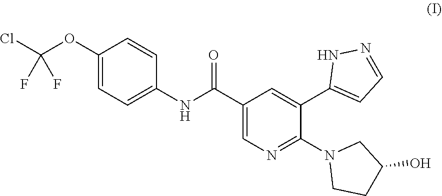 [SMILES: O=C(Nc1ccc(OC(F)(F)Cl)cc1)c1cnc(N2CC[C@@H](O)C2)c(-c2ccn[nH]2)c1]

or Compound I, preparation of the Compound I, and pharmaceutical compositions of Compound I are originally described in WO 2013/171639 A1 as Example 9. Compound I is also known as (R)-N-(4-(chlorodifluoromethoxy)phenyl)-6-(3-hydroxypyrrolidin-1-yl)-5-(1H-pyrazol-5-yl)nicotinamide, or asciminib. (R)-N-(4-(chlorodifluoromethoxy)phenyl)-6-(3-hydroxypyrrolidin-1-yl)-5-(1H-pyrazol-5-yl)nicotinamide (left structure, below) is a tautomer of (R)-N-(4-(chlorodifluoromethoxy)phenyl)-6-(3-hydroxypyrrolidin-1-yl)-5-(1H-pyrazol-3-yl)nicotinamide (right structure, below) and vice versa:

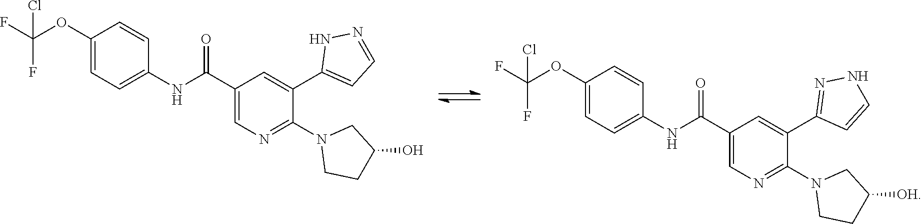 [SMILES: O=C(Nc1ccc(OC(F)(F)Cl)cc1)c1cnc(N2CC[C@@H](O)C2)c(-c2ccn[nH]2)c1 ; O=C(Nc1ccc(OC(F)(F)Cl)cc1)c1cnc(N2CC[C@@H](O)C2)c(-c2cc[nH]n2)c1]

WO 2013/171639 A1 describes Compound I as being useful in treating diseases which respond to inhibition of the tyrosine kinase enzymatic activity of the Abelson protein (ABL1), the Abelson-related protein (ABL2) and related chimeric proteins, in particular BCR-ABL1.

Further provided herein are specific dose regimens for the methods or uses of a BCR-ABL inhibitor, in particular Compound I, described herein.

Additionally described herein are pharmaceutical combinations comprising a) Compound I and b) at least one further therapeutic agent, optionally in the presence of a pharmaceutically acceptable carrier, for use in the treatment of a BCR-ABL-mediated disease or disorder and pharmaceutical compositions comprising them. Preferably, the one further therapeutic agent is selected from imatinib, nilotinib, dasatinib, bosutinib, ponatinib and bafetinib; more preferably, imatinib. In one embodiment, the pharmaceutical combination is administered together with food, preferably a low-fat meal.

Further features and advantages of the described methods and uses will become apparent from the following detailed description.

### BRIEF DESCRIPTION OF THE DRAWINGS

FIG. 1 shows an in vitro flux study assessing the impact of varying concentration of bile components (imitating fasted (FaSSIF) and fed (FeSSIF) intestinal conditions) on the (A) dissolution and (B) permeation through an artificial lipid membrane of asciminib (2×20 mg film-coated tablets) in Example 1.

FIG. 2 provides a schematic overview of the treatment protocol detailed in Example 2.

FIG. 3 shows the arithmetic mean (SD) plasma concentration-time profiles for (A) asciminib and (B) imatinib with asciminib alone and asciminib+imatinib (DDI group).

FIG. 4 shows the arithmetic mean (SD) plasma concentration-time profiles of asciminib when administered under fasted, low-fat meal, and high-fat meal conditions (FE group).

### DETAILED DESCRIPTION

Compound I is currently being investigated in patients with CML as a single agent and as TKI-combination regimens with the established CML treatments imatinib, nilotinib or dasatinib in a Phase 1, first-in-human, dose-finding study (NCT02081378), as a single agent in a Phase 3, randomized, controlled study (ASCEMBL; NCT03106779), and as an add-on therapy to front-line imatinib in a Phase 2 study in patients who had not achieved a deep molecular response (defined as BCR-ABL1 transcript values of ≤0.01% on the international reporting scale [IS]) with ≤1 years of frontline imatinib (ASC4MORE; NCT03578367).

For single-agent Compound I, the recommended Phase 3 dose in patients with CML was established as 40 mg twice daily (fasted state; maximum tolerated dose was not reached with doses up to 200 mg twice daily in patients with CML). In the Phase 3 ASCEMBL study in patients with CML in chronic phase (not harboring the BCR-ABL1 T315I mutation) who had been previously treated with ≥2 prior TKIs, Compound I monotherapy (40 mg twice daily) demonstrated statistically significant and clinically meaningful superiority vs the ATP-competitive BCR-ABL1 inhibitor bosutinib (500 mg once daily) for the primary endpoint major molecular response (BCR-ABL1≤0.1% on IS) at 24 weeks (25.5% vs 13.2%; difference 12.2%; 95% CI, 2.19-22.3: P=0.029), with a favorable safety profile.

The combination of asciminib+imatinib was well-tolerated in the first-in-human Phase 1 study in patients with CML who were resistant or intolerant to other TKIs, and demonstrated promising preliminary efficacy. Preliminary PK analyses in this population assessing a potential drug-drug interaction (DDI) between asciminib and imatinib found that asciminib 40 mg once daily plus imatinib 400 mg once daily with food (owing to better tolerability of imatinib when taken with food) provided comparable asciminib exposure to that achieved with the recommended asciminib monotherapy dose in the fasted state. The first-in-human study used initial asciminib tablet formulations, which had been shown to result in a moderate decrease in asciminib bioavailability when taken with food compared with the fasted state (30-31% decreased exposure with a low-fat meal; 63-64% decreased exposure with a high-fat meal). For commercial use of Compound I, a slightly modified tablet formulation (final marketed image [FMI]) was developed to ensure scalability to commercial batch size. Hence, further investigation was warranted to better characterize the DDI between imatinib and Compound I and establish the food effect (FE) on the marketed tablet formulation of Compound I.

Herein are described methods of treating a BCR-ABL-mediated disease or disorder by administering to a subject in need thereof an effective amount of Compound I or pharmaceutically acceptable salts thereof.

Accordingly, in one aspect provided is a method of treating a BCR-ABL-mediated disease or disorder comprising administering to a subject in need thereof an effective amount of Compound I or pharmaceutically acceptable salts thereof, without food. In a further aspect, the administration of Compound I or pharmaceutically acceptable salts thereof with food results in a decrease in bioavailability in the subject as compared to the administration of Compound I or pharmaceutically acceptable salts thereof without food. In a further aspect, the AUC is decreased by 30% to 60% with the administration of Compound I or pharmaceutically acceptable salts thereof with food as compared to without food.

In another aspect provided is a method of treating a BCR-ABL-mediated disease or disorder comprising administering to a subject in need thereof a pharmaceutical combination comprising an effective amount of Compound I or pharmaceutically acceptable salts thereof and an effective amount of at least one further therapeutic agent, optionally in the presence of a pharmaceutically acceptable carrier, for use in the treatment of a BCR-ABL-mediated disease or disorder and pharmaceutical compositions comprising them. Preferably, the one further therapeutic agent is selected from imatinib, nilotinib, dasatinib, bosutinib, ponatinib and bafetinib; more preferably, imatinib. In a further aspect, the pharmaceutical combination is administered together with food, preferably a low-fat meal. In a further aspect, the administration of the pharmaceutical combination with food results in about a 2-fold increase in systemic exposure of Compound I (AUCinf and AUClast) and about a 1.6-fold increase in Cmax of Compound I as compared to Compound I taken with food.

### Definitions

In order that the present document may be more readily understood, certain terms are first defined. Additional definitions are set forth throughout this document.

As used herein, the term “comprising” encompasses “including” as well as “consisting of” e.g., a composition “comprising” X may consist exclusively of X or may include something additional, e.g., X+Y.

As used herein, the articles “a” and “an” refer to one or to more than one (e.g., to at least one) of the grammatical object of the article.

The term “or” is used herein to mean, and is used interchangeably with, the term “and/or”, unless context clearly indicates otherwise.

The term “about” in relation to a reference numerical value and its grammatical equivalents as used herein can include the numerical value itself and a range of values plus or minus 10% from that numerical value. For example, the amount “about 10” includes 10 and any amounts from 9 to 11. For example, the term “about” in relation to a reference numerical value can also include a range of values plus or minus 10%, 9%, 8%, 7%, 6%, 5%, 4%, 3%, 2%, or 1% from that value. In some cases, the numerical value described throughout can be “about” that numerical value even without specifically mentioning the term “about.”

As used herein, the term “baseline” refers to a subject's state or the degree of a condition, e.g., a disease, or one or more parameters associated with the state of a patient, observed before treatment, e.g., before administration of a compound, e.g., before administration of an Compound I optionally in combination with at least one further therapeutic agent, according to the described methods and uses.

As used herein, the term “administering” in relation to a compound, e.g., the Compound I optionally in combination with at least one further therapeutic agent, is used to refer to delivery of that compound by any route of delivery. Such delivery may be, for example, an intravenous administration or oral administration. Such delivery may also be, for example, a subcutaneous administration.

As used herein, the terms “administered with food” or “with food” or “fed state” or “fed conditions or “fed” refers to the condition of having consumed food, for example, any food product, solid or liquid, with caloric content. The term “food” refers to, for example, food as defined in section 201(f) of the Federal Food, Drug, and Cosmetic Act and includes raw materials and ingredients. Preferably, the food is a solid food with sufficient bulk and fat content that it is not rapidly dissolved and absorbed in the stomach. The dosage of the Compound I may be administered to a subject, for example, between thirty (30) minutes prior to eating food to about two (2) hours after consumption. In embodiments, food has been consumed for about 10 hours, about 8 hours, about 6 hours, about 4 hours, about 2 hours, about 1 hour, or about 30 minutes prior to administration of Compound I. Preferably, administration of Compound I may occur immediately after consuming food up to about thirty (30) minutes after consumption.

As used herein, the term “without food” or “fasted state” or “fasted conditions” or “fasted” refers to, for example, the condition of not having consumed solid food for about or greater than one (1) hour prior to until about or greater than two (2) hours after such consumption. In some embodiments, food has not been consumed for about 10 hours, about 8 hours, about 6 hours, about 4 hours, about 2 hours, about 1 hour, or about 30 minutes prior to administration of Compound I. In some embodiments, food has not been consumed for about 10 hours, about 8 hours, about 6 hours, about 4 hours, about 2 hours, about 1 hour, or about 30 minutes after administration of Compound I.

As used herein, the term “low-fat meal” refers to the definition by the U.S. Food and Drug Administration in the draft guidance on Assessing the Effects of Food on Drugs in INDs and NDAs (FDA 2019) (see also Assessing the Effects of Food on Drugs in Investigational New Drug Applications and New Drug Applications-Clinical Pharmacology Considerations; Draft Guidance for Industry; Availability, 84 Fed. Reg. 6151 (Feb. 26, 2019)). An example of a low-fat meal would be a meal with less than 20% fat and about 400 calories.

As used herein, the term “administered with a low-fat meal” or “with a low-fat meal” is defined to mean the condition of having consumed a low-fat meal together with administration of Compound I within a certain time prior to administration of Compound I. The dosage of the Compound I may be administered to a subject, for example, between thirty (30) minutes prior to eating a low-fat meal to about two (2) hours after consumption. In embodiments, the low-fat meal has been consumed for about 10 hours, about 8 hours, about 6 hours, about 4 hours, about 2 hours, about 1 hour, or about 30 minutes prior to administration of Compound I. Preferably, administration of Compound I may occur immediately after consuming a low-fat meal up to about thirty (30) minutes after consumption.

As used herein, the term “pharmaceutically acceptable” means a nontoxic material that does not substantially interfere with the effectiveness of the biological activity of the active ingredient(s).

As used herein, the term “patient” is used interchangeably with the term “subject” and includes any human or nonhuman animal. The term “nonhuman animal” includes all vertebrates, e.g., mammals and non-mammals, such as nonhuman primates, sheep, dogs, cats, horses, cows, chickens, amphibians, reptiles, etc. In a specific embodiment, the compositions, methods, and uses described herein are in reference to a human patient or human subject.

As used herein, a subject is “in need of” a treatment if such subject who is afflicted with the condition (i.e., disease, disorder, or syndrome) of interest and who would benefit biologically, medically, or in quality of life from such treatment.

As used herein, the term “BCR-ABL-mediated disease or disorder” is disease or disorder associated with abnormally activated kinase activity of wild-type ABL1, including non-malignant diseases or disorders, such as CNS diseases in particular neurodegenerative diseases (for example Alzheimer's, Parkinson's diseases), motoneuroneuron diseases (amyotophic lateral sclerosis), muscular dystrophies, autoimmune and inflammatory diseases (diabetes and pulmonary fibrosis), viral infections, prion diseases. Preferably, the disease or disorder is a leukemia selected from chronic myeloid leukemia (CML), acute lymphoblastic leukemia (ALL), and acute myeloid leukemia (AML).

GLEEVEC® (imatinib mesylates) is indicated for the treatment of patients with KIT (CD 117)-positive unresectable and/or metastatic malignant gastrointestinal stromal tumors (GIST). It is also indicated to treat adult patients following complete gross resection of KIT (CD117)-positive GIST. It is also indicated for the treatment of newly diagnosed adult and pediatric patients with Philadelphia chromosome—positive chronic myeloid leukemia (Ph+ CML) in the chronic phase and patients with Ph+ CML in blast crisis (BC), accelerated phase (AP), or in the chronic phase (CP) after failure of interferon-alpha therapy. It can also be used as a targeted medicine for the treatment of the following rare disorders with limited treatment options: relapsed or refractory Philadelphia chromosome-positive acute lymphoblastic leukemia (Ph+ ALL); myelodysplastic/myeloproliferative diseases (MDS/MPD) associated with platelet-derived growth factor receptor (PDGFR) gene rearrangements; aggressive systemic mastocytosis (ASM) without the D816V c-KIT mutation or with c-KIT mutational status unknown; hypereosinophilic syndrome/chronic eosinophilic leukemia (HES/CEL) with the FIP1L1-PDGFRu fusion kinase (mutational analysis or FISH demonstration of CHIC2 allele deletion) and for patients with HES and/or CEL who are FIP1L1-PDGFRu fusion kinase negative or unknown; and unresectable, recurrent, and/or metastatic dermatofibrosarcoma protuberans (DFSP).

TASIGNA® (nilotinib) is indicated for the treatment of adult patients with newly diagnosed Philadelphia chromosome-positive chronic myeloid leukemia (Ph+ CML) in chronic phase It can be used to treat adults who are no longer benefiting from, or are intolerant to other treatments, including imatinib (GLEEVEC®), or have taken other treatments, including imatinib (GLEEVEC) but cannot tolerate them.

SPRYCEL® (dasatinib) is a prescription medicine used to treat adults who have newly diagnosed Philadelphia chromosome-positive (Ph+) chronic myeloid leukemia (CML) in chronic phase and to treat adults who are no longer benefitting or are intolerant to other treatments, as well as for patients with ALL.

BOSULIF® (bosutinib) is a prescription medicine used to treat adults who have newly diagnosed Philadelphia chromosome-positive (Ph+) chronic myeloid leukemia (CML) in chronic phase and to treat adults who are no longer benefitting or are intolerant to other treatments, as well as for patients with ALL.

The term “treatment” comprises, for example, the therapeutic administration of Compound I, or a pharmaceutically acceptable salt thereof, or the combination of Compound I, or a pharmaceutically acceptable salt thereof, and at least one further therapeutic agent, as described herein to a warm-blooded animal, in particular a human being, in need of such treatment with the aim to cure the disease or to have an effect on disease regression or on the delay of progression of a disease. The terms “treat”, “treating” or “treatment” of any disease or disorder refers to ameliorating the disease or disorder (e.g. slowing or arresting or reducing the development of the disease or at least one of the clinical symptoms thereof), to preventing or delaying the onset or development or progression of the disease or disorder. More specifically, the one further therapeutic agent is selected from imatinib, nilotinib, dasatinib, bosutinib, ponatinib and bafetinib; more preferably, imatinib.

The term “treat”, “treating”, or “treatment” includes therapeutic treatments, prophylactic treatments and applications in which one reduces the risk that a subject will develop a disorder or other risk factor. Treatment does not require the complete curing of a disorder and encompasses the reduction of the symptoms or underlying risk factors.

The term “treating” includes the administration of a compound, e.g., the Compound I optionally in combination with at least one further therapeutic agent, to prevent or delay the onset of the symptoms, complications, or biochemical indicia of a disease, condition, disorder, or syndrome (e.g., a BCR-ABL-mediated disease or disorder), alleviating the symptoms or arresting or inhibiting further development or manifestation of the disease, condition, disorder, or syndrome.

As used herein, term “excipient” or “pharmaceutically acceptable excipient” means a pharmaceutically-acceptable material, composition, or vehicle, such as a liquid or solid filler, diluent, carrier, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Remington: The Science and Practice of Pharmacy, 21st ed.; Lippincott Williams & Wilkins: Philadelphia, P A, 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, F L, 2009.

As used herein, the term “BCR-ABL inhibitor” is a compound that inhibits the tyrosine kinase enzymatic activity of the Abelson protein (ABL1), the Abelson-related protein (ABL2) and related chimeric proteins, in particular BCR-ABL1.

As used herein, “Compound of formula I,” or “Compound I,” are used interchangeably and mean a compound that has the structure shown below, and can be synthesized using procedures known in the art and described in WO2013/171639, incorporated by reference in its entirety

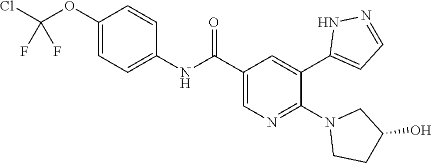 [SMILES: O=C(Nc1ccc(OC(F)(F)Cl)cc1)c1cnc(N2CC[C@@H](O)C2)c(-c2ccn[nH]2)c1]

Any chemical formula given herein is also intended to represent unlabeled forms as well as isotopically labeled forms of the compounds. Isotopically labeled compounds have structures depicted by the formulae given herein except that one or more atoms are replaced by an atom having a selected atomic mass or mass number. Isotopes that can be incorporated into compounds of the disclosure include, for example, isotopes of hydrogen, carbon, nitrogen, and oxygen, such as ³H, ¹¹C, ¹³C, ¹⁴C, and ¹⁵N. Accordingly, it should be understood that methods of the present invention can or may involve compounds that incorporate one or more of any of the aforementioned isotopes, including for example, radioactive isotopes, such as ³H and ¹⁴C, or those into which non-radioactive isotopes, such as ²H and 1³C are present. Such isotopically labelled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. Isotopically-labeled compounds can generally be prepared by conventional techniques known to those skilled in the art, e.g., using an appropriate isotopically-labeled reagents in place of the non-labeled reagent previously employed. The present invention encompasses embodiments that include all pharmaceutically acceptable salts of the compounds useful according to the invention provided herein. As used herein, “pharmaceutically acceptable salt” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts of the parent compound formed, for example, from non-toxic inorganic or organic acids. The pharmaceutically acceptable salts can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. Lists of suitable salts are found in Remington's Pharmaceutical Sciences, 17^{th} ed., Mack Publishing Company, Easton, Pa., 1985, p. 1418 and Journal of Pharmaceutical Science, 66, 2 (1977), each of which is incorporated herein by reference in its entirety. For example, preferred pharmaceutically acceptable salts include, but are not limited to, mineral or organic acid salts of basic residues such as amines. For example, the salt can be a hydrochloride salt.

The phrase “pharmaceutically acceptable” as employed herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Unless otherwise indicated, as used here, the “dose” or amount of BCR-ABL inhibitor, e.g., Compound I, refers to the amount of the free base or free acid form of the compound. For salt forms of the BCR-ABL inhibitor, the actual amount will be adjusted based on the salt form used.

An “effective amount” refers to an amount sufficient to effect beneficial or desired results. For example, a therapeutic amount is one that achieves the desired therapeutic effect. This amount can be the same or different from a prophylactically effective amount, which is an amount necessary to prevent onset of disease, condition, disorder, or syndrome or related symptoms. An effective amount can be administered in one or more administrations, applications or dosages. A “therapeutically effective amount” of a therapeutic compound (i.e., an effective dosage) depends on the therapeutic compounds selected. The compositions can be administered from one or more times per day to one or more times per week, and also include less frequent administration, e.g., as described herein. The skilled artisan will appreciate that certain factors may influence the dosage and timing required to effectively treat a subject, including but not limited to the severity of the disease, condition, disorder, or syndrome, previous treatments, the general health and/or age of the subject, and other concurrent diseases, conditions, disorders, or syndromes. Moreover, treatment of a subject with a therapeutically effective amount of the therapeutic compounds described herein can include a single treatment or a series of treatments.

As used herein, the term “therapeutically effective amount” of the compound described herein refers to an amount of the compound that will elicit the biological or medical response of a subject, for example, ameliorate symptoms, alleviate conditions, slow or delay disease progression, or prevent a disease, condition, disorder, manifestation or syndrome, etc. In one non-limiting embodiment, the term “a therapeutically effective amount” refers to the amount of the compound described herein that, when administered to a subject, is effective to at least partially alleviating, inhibiting, preventing and/or ameliorating an BCR-ABL mediated disease or disorder (e.g., a leukemia selected from chronic myeloid leukemia (CML), acute lymphoblastic leukemia (ALL), and acute myeloid leukemia (AML).

As herein defined, “combination” refers to either a fixed combination in one unit dosage form (e.g., capsule, tablet, sachet or vial), free (i.e., non-fixed) combination, or a kit of parts for the combined administration where an Compound I and the one or more additional therapeutic agents may be administered independently at the same time or separately within time intervals, especially where these time intervals allow that the combination partners show a cooperative, e.g., synergistic effect.

As used herein, “add-on” or “add-on therapy” means an assemblage of therapeutic agents for use in therapy, wherein the subject receiving the therapy begins a first treatment regimen of one or more therapeutic agents prior to beginning a second treatment regimen of one or more different therapeutic agents in addition to the first treatment regimen, so that not all of the therapeutic agents used in the therapy are started at the same time. For example, adding BCR-ABL inhibitor such as Compound I to a patient already receiving at least one further therapeutic agent such as imatinib, nilotinib, dasatinib, bosutinib, ponatinib and bafetinib. It was shown that a higher MR^{4.5} rate (whereas BCR-ABL1/ABL ratio of ≤0.0032% identifies a 4.5-log reduction (MR^{4.5})) at week 48 can be achieved with asciminib add-on therapy vs imatinib and nilotinib. Asciminib add-on therapy was tested in a phase 2, multicenter, open-label, randomized study of asciminib add-on to 1L imatinib vs continued imatinib vs switch to nilotinib (NCT03578367). Cumulative MR4.5 rates occurred earlier and were higher with asciminib add-on therapy than with imatinib and nilotinib. MR^{4.5} rates by week 48 were 19.0% with asciminib 40 mg QD add-on, 28.6% with asciminib 60 mg QD add-on, 0% with imatinib, and 14.3% with nilotinib.

The terms “co-administration” or “combined administration” or the like as utilized herein are meant to encompass administration of an additional therapeutic agent to a single subject in need thereof (e.g., a subject), and the additional therapeutic agent are intended to include treatment regimens in which the Compound I and additional therapeutic agent are not necessarily administered by the same route of administration and/or at the same time. Each of the components of the presently described combination may be administered simultaneously or sequentially and in any order. Co-administration comprises simultaneous, sequential, overlapping, interval, and/or continuous administrations and any combination thereof.

The term “pharmaceutical combination” as used herein means a pharmaceutical composition that results from the combining (e.g., mixing) of more than one active ingredient and includes both fixed and free combinations of the active ingredients.

The term “fixed combination” means that the active ingredients are administered to a subject simultaneously in the form of a single entity or dosage.

The term “free combination” (non-fixed combination) means that the active ingredients as defined herein are administered to a subject as separate entities either simultaneously, concurrently or sequentially with no specific time limits, and in any order, wherein such administration provides therapeutically effective levels of the compounds in the subject's body. In particular, reference to the combination comprising a) a Compound I and b) at least one additional therapeutic agent as used herein (e.g., in any of the embodiments or in any of the claims herein), refers to a “non-fixed combination” and may be administered independently at the same time or separately within time intervals.

By “simultaneous administration”, it is meant that the active ingredients as defined herein, are administered on the same day. The active ingredients can be administered at the same time (for fixed or free combinations), or one at a time (for free combinations).

The term “sequential administration”, may mean that during a period of two or more days of continuous co-administration only one of active ingredients as herein defined, is administered on any given day.

By “overlapping administration”, it is meant that during a period of two or more days of continuous co-administration, there is at least one day of simultaneous administration and at least one day when only one of active ingredients as herein defined, is administered.

By “continuous administration”, it is meant a period of co-administration without any void day.

The continuous administration may be simultaneous, sequential, or overlapping, as described above.

The term “dose” refers to a specified amount of a drug administered at one time. The dose could, for example, be declared on a product package or in a product information leaflet.

### Enumerated Embodiments (embodiments 1 to 21)

Embodiment 1: A method of treating a BCR-ABL mediated disease or disorder in a patient in need thereof, comprising administering a pharmaceutical combination comprising (i) a therapeutically effective amount of asciminib or a pharmaceutically acceptable salt thereof and (ii) a therapeutically effective amount of at least one further therapeutic agent; wherein the pharmaceutical combination is administered together with food.

Embodiment 2: The method of embodiment 1, wherein the BCR-ABL mediated disease or disorder is a leukemia selected from chronic myeloid leukemia (CML), acute lymphoblastic leukemia (ALL), and acute myeloid leukemia (AML).

Embodiment 3: The method embodiments 1 or 2, wherein asciminib or a pharmaceutically acceptable salt thereof is used in add-on combination therapy to the one further therapeutic agent.

Embodiment 4: The method according to any one of embodiments 1 to 3, wherein asciminib or a pharmaceutically acceptable salt thereof is administered to the patient at a total daily dose of about 40 mg or 60 mg in a single dose.

Embodiment 5: The method according to any one of embodiments 1 to 4, wherein the one further therapeutic agent is selected from imatinib, nilotinib, dasatinib, bosutinib, ponatinib and bafetinib.

Embodiment 6: The method of claim 5, wherein the one further therapeutic agent is imatinib.

Embodiment 7: The method according to any one of embodiments 1 to 6, wherein imatinib is administered to the patient at a total daily dose of about 400 mg in a single dose.

Embodiment 8: The method according to any one of embodiments 1 to 7, wherein the food is a low-fat meal.

Embodiment 9: The method according to any one of embodiments 1 to 8, wherein the pharmaceutical combination is administered together sequentially or simultaneously.

Embodiment 10: A method of treating a BCR-ABL mediated disease or disorder in a patient in need thereof, comprising administering a total daily dose of about 40 mg or 60 mg of asciminib or a pharmaceutically acceptable salt thereof, in a single dose and a total daily dose of about 400 mg of imatinib in a single dose, wherein the single dose of asciminib or a pharmaceutically acceptable salt thereof, and the single dose of imatinib are administered together with a low-fat meal.

Embodiment 11: The method according to embodiment 10, wherein the dose of asciminib or a pharmaceutically acceptable salt thereof and the dose of imatinib are administered simultaneously.

Embodiment 12: The method according to embodiment 10 or 11 wherein asciminib or a pharmaceutically acceptable salt thereof is used in add-on combination therapy to imatinib.

Embodiment 13: A method of treating a BCR-ABL mediated disease or disorder in a patient in need thereof, comprising administering a therapeutically effective amount of asciminib or a pharmaceutically acceptable salt thereof without food.

Embodiment 14: The method according to embodiment 13, wherein the administration of asciminib or a pharmaceutically acceptable salt thereof with food results in a decrease in bioavailability in the subject as compared to the administration of asciminib or a pharmaceutically acceptable salt thereof without food.

Embodiment 15: The method according to embodiments 13 or 14, wherein the BCR-ABL mediated disease or disorder is a leukemia selected from chronic myeloid leukemia (CML), acute lymphoblastic leukemia (ALL), and acute myeloid leukemia (AML).

Embodiment 16: The method according to any one of embodiments 13 to 15, wherein asciminib or a pharmaceutically acceptable salt thereof is administered to the patient at a total daily dose of about 80 mg in divided doses. A method of treating a BCR-ABL mediated disease or disorder in a patient in need thereof, comprising administering a therapeutically effective amount of asciminib or a pharmaceutically acceptable salt thereof without food.

Embodiment 17: A method of treating a BCR-ABL mediated disease or disorder in a patient in need thereof, comprising administering (i) a therapeutically effective amount of asciminib or a pharmaceutically acceptable salt thereof and (ii) a therapeutically effective amount of at least one further therapeutic agent a patient, wherein asciminib or a pharmaceutically acceptable salt thereof is used in add-on combination therapy to the one further therapeutic agent.

Embodiment 18: A method according to embodiment 17, wherein the one further therapeutic agent is selected from imatinib, nilotinib, dasatinib, bosutinib, ponatinib and bafetinib.

Embodiment 19: A method according to embodiment 17 or 18, wherein the one further therapeutic agent is imatinib.

Embodiment 20: A method according to any of embodiments 17 to 19 wherein asciminib or a pharmaceutically acceptable salt thereof is administered to the patient at a total daily dose of about 40 mg or 60 mg in a single dose.

Embodiment 21: A method according to any of embodiments 17 to 20, wherein imatinib is administered to the patient at a total daily dose of about 400 mg in a single dose.

### Uses and Methods

Various embodiments of the methods and uses described herein are included below and elsewhere in the document. It will be recognized that features specified in each embodiment may be combined with other specified features to provide further embodiments:

In one embodiment, provided herein is a method of treating a BCR-ABL mediated disease or disorder in a subject in need thereof, comprising administering an effective amount of Compound I, or a pharmaceutically acceptable salt thereof, without food. In one embodiment, provided herein is Compound I or a pharmaceutically acceptable salt thereof for use in treating a BCR-ABL mediated disease or disorder in a subject in need thereof, without food. In some embodiments, provided herein is the use of Compound I, for the manufacture of a medicament for the treatment of a BCR-ABL mediated disease or disorder, without food.

In another embodiment, provided herein is a method of treatment or reducing the symptoms of a a leukemia selected from chronic myeloid leukemia (CML), acute lymphoblastic leukemia (ALL), and acute myeloid leukemia (AML) in a subject in need thereof, comprising administering an effective amount of Compound I, or a pharmaceutically acceptable salt thereof, without food. In one embodiment, provided herein is Compound I or a pharmaceutically acceptable salt thereof for use in treating a leukemia selected from chronic myeloid leukemia (CML), acute lymphoblastic leukemia (ALL), and acute myeloid leukemia (AML) in a subject in need thereof, without food. In some embodiments, provided herein is the use of Compound I, for the manufacture of a medicament for the treatment of a leukemia selected from chronic myeloid leukemia (CML), acute lymphoblastic leukemia (ALL), and acute myeloid leukemia (AML), without food.

In another embodiment, provided herein is a method of treating a BCR-ABL mediated disease or disorder in a subject in need thereof, comprising administering a pharmaceutical combination of (i) an effective amount of Compound I, or a pharmaceutically acceptable salt thereof, and (ii) an effective amount of at least one further therapeutic agent. In one embodiment, provided herein is a pharmaceutical combination of Compound I or a pharmaceutically acceptable salt thereof, and an least one further therapeutic agent, for use in treating a BCR-ABL mediated disease or disorder in a subject in need thereof. In some embodiments, provided herein is the use of a pharmaceutical combination of Compound I or a pharmaceutically acceptable salt thereof, and an least one further therapeutic agent, for the manufacture of a medicament for the treatment of a BCR-ABL mediated disease or disorder.

In another embodiment, provided herein is a method of treating a leukemia selected from chronic myeloid leukemia (CML), acute lymphoblastic leukemia (ALL), and acute myeloid leukemia (AML) in a subject in need thereof, comprising administering a pharmaceutical combination of (i) an effective amount of Compound I, or a pharmaceutically acceptable salt thereof, and (ii) an effective amount of at least one further therapeutic agent. In one embodiment, provided herein is a pharmaceutical combination of Compound I or a pharmaceutically acceptable salt thereof, and an least one further therapeutic agent, for use in treating a leukemia selected from chronic myeloid leukemia (CML), acute lymphoblastic leukemia (ALL), and acute myeloid leukemia (AML) in a subject in need thereof. In some embodiments, provided herein is the use of a pharmaceutical combination of Compound I or a pharmaceutically acceptable salt thereof, and an least one further therapeutic agent, for the manufacture of a medicament for the treatment of a leukemia selected from chronic myeloid leukemia (CML), acute lymphoblastic leukemia (ALL), and acute myeloid leukemia (AML).

In another embodiment, provided herein is a method of treating a leukemia selected from chronic myeloid leukemia (CML), acute lymphoblastic leukemia (ALL), and acute myeloid leukemia (AML) in a subject in need thereof, comprising administering a pharmaceutical combination of (i) an effective amount of Compound I, or a pharmaceutically acceptable salt thereof, and (ii) an effective amount of at least one further therapeutic agent selected from imatinib, nilotinib, dasatinib, bosutinib, ponatinib and bafetinib, with food, preferably a low-fat meal. In one embodiment, provided herein is a pharmaceutical combination of Compound I or a pharmaceutically acceptable salt thereof, and an least one further therapeutic agent selected from imatinib, nilotinib, dasatinib, bosutinib, ponatinib and bafetinib, for use in treating a leukemia selected from chronic myeloid leukemia (CML), acute lymphoblastic leukemia (ALL), and acute myeloid leukemia (AML) in a subject in need thereof, with food, preferably a low-fat meal. In some embodiments, provided herein is the use of a pharmaceutical combination of Compound I or a pharmaceutically acceptable salt thereof, and an least one further therapeutic agent is selected from imatinib, nilotinib, dasatinib, bosutinib, ponatinib and bafetinib, for the manufacture of a medicament for the treatment of a leukemia selected from chronic myeloid leukemia (CML), acute lymphoblastic leukemia (ALL), and acute myeloid leukemia (AML), with food, preferably a low-fat meal.

In any of the embodiments described herein, Compound I or a pharmaceutically acceptable salt thereof may be administered to the patient at a total daily dose of about 20 mg to about 400 mg, as measured in the non-salt equivalents, in single or divided doses. In particular embodiments, Compound I is administered to the patient at a total daily dose of about 80 mg in single or divided doses. In yet particular embodiments, Compound I is administered to the patient at a dose of about 40 mg twice daily.

In some embodiments, provided herein is a pharmaceutical composition comprising Compound I or a pharmaceutically acceptable salt thereof, and at least one pharmaceutically acceptable excipient. In particular embodiments, the pharmaceutical composition is a tablet. In yet particular embodiments, the pharmaceutical composition is administered as a whole or crushed tablet. In some embodiments, the pharmaceutical composition includes about 5 mg, about 10 mg, about 15 mg, about 20 mg, about 25 mg, about 30 mg, about 35 mg, about 40 mg, about 45 mg, about 50 mg, about 60 mg, about 70 mg, about 80 mg, about 90 mg, or about 100 mg in each unit dose.

Provided herein is a pharmaceutical composition comprising Compound I or a pharmaceutically acceptable salt thereof, for use in any of the embodiments described herein.

In any of embodiments described herein, Compound I or a pharmaceutically acceptable salt thereof is administered to a subject in need thereof orally. In some embodiments, Compound I is in the form of a table that is administered either whole or subdivided, i.e., crushed prior to administration.

In particular embodiments, for example when patients are unable to swallow, Compound I may be administered via a nasogastric tube.

### Pharmaceutical Compositions

Compound I may be used as a pharmaceutical composition when combined with a pharmaceutically acceptable carrier. Such a composition may contain, in addition to Compound I, carriers, various diluents, fillers, salts, buffers, stabilizers, solubilizers, and other known materials. The characteristics of the carrier will depend on the route of administration. The pharmaceutical compositions for use in the compositions, uses, and methods described herein may also contain at least one or more additional therapeutic agents for treatment of the particular targeted disorder, disease, condition, or syndrome. Such additional factors and/or agents may be included in the pharmaceutical composition to produce a synergistic effect with Compound I.

In specific embodiments, the Compound I can be administered in combination with one or more conventional pharmaceutical excipients. Pharmaceutically acceptable excipients include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens, poloxamers or other similar polymeric delivery matrices, serum proteins, such as human serum albumin, buffer substances such as phosphates, tris, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium-chloride, zinc salts, colloidal silica, magnesium trisilicate, polyvinyl pyrrolidone, cellulose-based substances, polyethylene glycol, sodium carboxymethyl cellulose, polyacrylates, waxes, polyethylene-polyoxypropylene-block polymers, and wool fat. Cyclodextrins such as α-, β, and γ-cyclodextrin, or chemically modified derivatives such as hydroxyalkylcyclodextrins, including 2- and 3-hydroxypropyl-β-cyclodextrins, or other solubilized derivatives can also be used to enhance delivery of compounds described herein. Dosage forms or compositions containing a chemical entity as described herein in the range of 0.005% to 100% with the balance made up from non-toxic excipient may be prepared. The contemplated compositions may contain 0.001%-100% of a chemical entity provided herein, in one embodiment 0.1-95%, in another embodiment 75-85%, in a further embodiment 20-80%. Actual methods of preparing such dosage forms are known, or will be apparent, to those skilled in this art; for example, see Remington: The Science and Practice of Pharmacy, 22^{nd} Edition (Pharmaceutical Press, London, U K. 2012).

### Routes of Administration and Composition Components

In some embodiments, the chemical entities described herein or a pharmaceutical composition thereof can be administered to subject in need thereof by any accepted route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal, nasogastric, oral, parenteral, percutaneous, peridural, rectal, respiratory (inhalation), subcutaneous, sublingual, submucosal, topical, transdermal, transmucosal, transtracheal, ureteral, urethral and vaginal. In certain embodiments, a preferred route of administration is parenteral (e.g., intratumoral).

Compositions can be formulated for parenteral administration, e.g., formulated for injection via the intravenous, intramuscular, sub-cutaneous, or even intraperitoneal routes. Typically, such compositions can be prepared as injectables, either as liquid solutions or suspensions; solid forms suitable for use to prepare solutions or suspensions upon the addition of a liquid prior to injection can also be prepared; and the preparations can also be emulsified. The preparation of such formulations will be known to those of skill in the art in light of the present disclosure.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The carrier also can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Intratumoral injections are discussed, e.g., in Lammers, et al., “Effect of Intratumoral Injection on the Biodistribution and the Therapeutic Potential of HPMA Copolymer-Based Drug Delivery Systems” Neoplasia. 2006, 10, 788-795.

In certain embodiments, the chemical entities described herein or a pharmaceutical composition thereof are suitable for local, topical administration to the digestive or GI tract, e.g., rectal administration. Rectal compositions include, without limitation, enemas, rectal gels, rectal foams, rectal aerosols, suppositories, jelly suppositories, and enemas (e.g., retention enemas).

Pharmacologically acceptable excipients usable in the rectal composition as a gel, cream, enema, or rectal suppository, include, without limitation, any one or more of cocoa butter glycerides, synthetic polymers such as polyvinylpyrrolidone, PEG (like PEG ointments), glycerine, glycerinated gelatin, hydrogenated vegetable oils, poloxamers, mixtures of polyethylene glycols of various molecular weights and fatty acid esters of polyethylene glycol Vaseline, anhydrous lanolin, shark liver oil, sodium saccharinate, menthol, sweet almond oil, sorbitol, sodium benzoate, anoxid SBN, vanilla essential oil, aerosol, parabens in phenoxyethanol, sodium methyl p-oxybenzoate, sodium propyl p-oxybenzoate, diethylamine, carbomers, carbopol, methyloxybenzoate, macrogol cetostearyl ether, cocoyl caprylocaprate, isopropyl alcohol, propylene glycol, liquid paraffin, xanthan gum, carboxy-metabisulfite, sodium edetate, sodium benzoate, potassium metabisulfite, grapefruit seed extract, methyl sulfonyl methane (MSM), lactic acid, glycine, vitamins, such as vitamin A and E and potassium acetate.

In certain embodiments, suppositories can be prepared by mixing the chemical entities described herein with suitable non-irritating excipients or carriers such as cocoa butter, polyethylene glycol or a suppository wax which are solid at ambient temperature but liquid at body temperature and therefore melt in the rectum and release the active compound. In other embodiments, compositions for rectal administration are in the form of an enema.

In other embodiments, the compounds described herein or a pharmaceutical composition thereof are suitable for local delivery to the digestive or GI tract by way of oral administration (e.g., solid or liquid dosage forms.).

Solid dosage forms for oral administration include capsules, tablets, pills, powders, and granules. In such solid dosage forms, the chemical entity is mixed with one or more pharmaceutically acceptable excipients, such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

In one embodiment, the compositions will take the form of a unit dosage form such as a pill or tablet and thus the composition may contain, along with a chemical entity provided herein, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives or the like. In another solid dosage form, a powder, marume, solution or suspension (e.g., in propylene carbonate, vegetable oils, PEG's, poloxamer 124 or triglycerides) is encapsulated in a capsule (gelatin or cellulose base capsule). Unit dosage forms in which one or more chemical entities provided herein or additional active agents are physically separated are also contemplated; e.g., capsules with granules (or tablets in a capsule) of each drug; two-layer tablets; two-compartment gel caps, etc. Enteric coated or delayed release oral dosage forms are also contemplated.

Other physiologically acceptable compounds include wetting agents, emulsifying agents, dispersing agents or preservatives that are particularly useful for preventing the growth or action of microorganisms. Various preservatives are well known and include, for example, phenol and ascorbic acid.

In certain embodiments the excipients are sterile and generally free of undesirable matter. These compositions can be sterilized by conventional, well-known sterilization techniques. For various oral dosage form excipients such as tablets and capsules sterility is not required. The USP/NF standard is usually sufficient.

In certain embodiments, solid oral dosage forms can further include one or more components that chemically and/or structurally predispose the composition for delivery of the chemical entity to the stomach or the lower GI; e.g., the ascending colon and/or transverse colon and/or distal colon and/or small bowel. Exemplary formulation techniques are described in, e.g., Filipski, K. J., et al., Current Topics in Medicinal Chemistry, 2013, 13, 776-802, which is incorporated herein by reference in its entirety.

Examples include upper-GI targeting techniques, e.g., Accordion Pill (Intec Pharma), floating capsules, and materials capable of adhering to mucosal walls.

Other examples include lower-GI targeting techniques. For targeting various regions in the intestinal tract, several enteric/pH-responsive coatings and excipients are available. These materials are typically polymers that are designed to dissolve or erode at specific pH ranges, selected based upon the GI region of desired drug release. These materials also function to protect acid labile drugs from gastric fluid or limit exposure in cases where the active ingredient may be irritating to the upper GI (e.g., hydroxypropyl methylcellulose phthalate series, Coateric (polyvinyl acetate phthalate), cellulose acetate phthalate, hydroxypropyl methylcellulose acetate succinate, Eudragit series (methacrylic acid-methyl methacrylate copolymers), and Marcoat). Other techniques include dosage forms that respond to local flora in the GI tract, Pressure-controlled colon delivery capsule, and Pulsincap.

Ocular compositions can include, without limitation, one or more of any of the following: viscogens (e.g., Carboxymethylcellulose, Glycerin, Polyvinylpyrrolidone, Polyethylene glycol); Stabilizers (e.g., Pluronic (triblock copolymers), Cyclodextrins); Preservatives (e.g., Benzalkonium chloride, ETDA, SofZia (boric acid, propylene glycol, sorbitol, and zinc chloride; Alcon Laboratories, Inc.), Purite (stabilized oxychloro complex; Allergan, Inc.)).

Topical compositions can include ointments and creams. Ointments are semisolid preparations that are typically based on petrolatum or other petroleum derivatives. Creams containing the selected active agent are typically viscous liquid or semisolid emulsions, often either oil-in-water or water-in-oil. Cream bases are typically water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation is generally a nonionic, anionic, cationic or amphoteric surfactant. As with other carriers or vehicles, an ointment base should be inert, stable, nonirritating and non-sensitizing.

In any of the foregoing embodiments, pharmaceutical compositions described herein can include one or more one or more of the following: lipids, interbilayer crosslinked multilamellar vesicles, biodegradeable poly(D,L-lactic-co-glycolic acid) [PLGA]-based or poly anhydride-based nanoparticles or microparticles, and nanoporous particle-supported lipid bilayers.

### Dosing Regimen and Modes of Administration

Dosage regimens are adjusted to provide the optimum desired response (e.g., a therapeutic response). Depending on the compound used, the targeted disease, condition, disorder, or syndrome and the relevant stages of the same, the dosing regimen, i.e., administered doses and/or frequency of the pharmaceutical composition comprising Compound I may vary. Depending on the compound used, the disease, condition, disorder, or syndrome and the relevant stages of the same, the dosing regimen, i.e., administered doses and/or frequency of the pharmaceutical combination comprising a) Compound I and b) at least one further therapeutic agent, may vary.

For administration of Compound I in the methods for treating a BCR-ABL mediated disease or disorder, the dosage ranges from about 0.0001 to about 100 mg/kg, and more usually about 0.01 to about 30 mg/kg, of the subject's body weight. In particular embodiments, Compound I is administered at a daily dose of about 20 mg to about 400 mg, about 40 mg to about 300 mg, about 80 mg to about 240 mg, about 40 mg to about 80 mg, about 80 mg. In particular embodiments, Compound I is administered at a daily dose of about 20 mg, about 40 mg, about 80 mg, or about 100 mg. In particular embodiments, Compound I is administered once a day. In other embodiments, Compound I is administered two, three, or four times a day. In preferred embodiments, Compound I is administered at a daily total dose of about 80 mg, administered once or in two divided doses. In some embodiments, Compound I is administered at a daily dose of 80 mg in two divided doses. In another embodiment, Compound I is administered at a daily dose of 40 mg once a day.

### Kits

Herein are also encompassed kits for use in the methods for treating or preventing cytokine release syndrome or cytokine storm syndrome, which may comprise Compound I in liquid or lyophilized form or a pharmaceutical composition comprising Compound I. Additionally, such kits may comprise a means for administering Compound I (e.g., a syringe and vial, a prefilled syringe, a prefilled pen) and instructions for use. These kits may contain additional therapeutic agents (described elsewhere herein), e.g., for delivery in combination with Compound I.

The phrase “means for administering” is used to indicate any available implement for systemically administering a drug to a patient, including, but not limited to, a dropper, a pre-filled syringe, a vial and syringe, an injection pen, an autoinjector, an i.v. drip and bag, a pump, etc. With such items, a patient may self-administer the drug (i.e., administer the drug on their own behalf), a caregiver may administer the drug to the patient, or a physician or other medical professional may administer the drug.

Each component of the kit is usually enclosed within an individual container, and all of the various containers are within a single package along with instructions for use.

It is to be understood that each embodiment may be combined with one or more other embodiments, to the extent that such a combination is consistent with the description of the embodiments. It is further to be understood that the embodiments provided above are understood to include all embodiments, including such embodiments as result from combinations of embodiments.

Other features, objects, and advantages of the described methods and uses will be apparent from the description and drawings, and from the claims.

### EXAMPLES

The following Example illustrates the methods and uses described herein. They are not, however, intended to limit the scope of the described methods and uses in any way. Other variants of the embodiment will be readily apparent to one of ordinary skill in the art and are encompassed by the appended claims.

### Example 1: Flux Analysis

To provide further information on the food effect (FE) of asciminib (ABL001), an experimental in vitro study was performed to determine the impact of bile components on the dissolution and permeation through an artificial lipid membrane of a 40 mg dose of asciminib.

### Method

The donor compartment of a United States Pharmacopeia, Method II (USP II) dissolution apparatus (Distek corporation; New Jersey, United States) was filled with 900 mL maleate buffer containing either low concentrations of bile salts (imitating fasted conditions; Fasted State Simulated Intestinal Fluid [FaSSIF]; 3 mM taurocholate/taurodeoxycholate, 0.2 mM phospholipids/lysophospholipids, pH 5.8), high concentrations of bile salts (imitating fed intestinal conditions; Fed State Simulated Intestinal Fluid [FeSSIF]; 10 mM taurocholate/taurodeoxycholate, 2 mM phospholipids/lysophospholipids, 0.8/5.0 mM oleate/glycerol monoleate, pH 6.0), or no bile salts (control; pH 6.5). FaSSIF and FeSSIF were prepared according to the instructions of the manufacturer (Biorelevant. Available at: Biorelevant.com (accessed March 2021). In each of the 3 set-ups, 2 film-coated tablets of asciminib 20 mg (to achieve 40 mg total dose) were added to the buffer, and maintained at 37° C. with constant stirring (100 rpm). A receiver compartment, sealed by a 0.45 m polyvinylidene fluoride membrane and an artificial lipid membrane, and containing 12 mL of a proprietary acceptor buffer to help solubilize poorly soluble molecules to high concentration (pION, Inc; Massachusetts, USA), was then inserted into the donor compartment. Dissolution and flux rates of asciminib were determined by measuring the concentrations of asciminib in the donor and receiver compartments using fiber optic probes. Two replicates per condition were performed.

### Discussion and Results

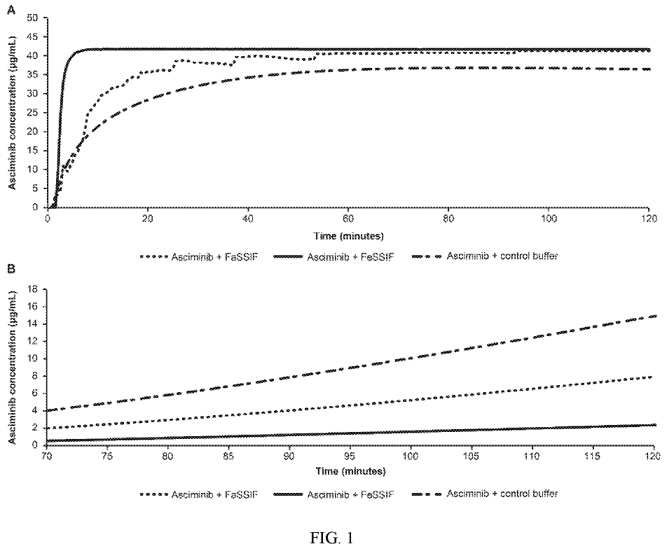

Fat intake has been shown to positively correlate with the level of excreted bile acids and thus, the different compositions of bile aimed to reflect fed and fasted conditions (Trefflich et al. Associations between Dietary Patterns and Bile Acids-Results from a Cross-Sectional Study in Vegans and Omnivores. Nutrients 2019; 12(1)). The dissolution rate of asciminib was fastest in a buffer containing a composition of bile salts, acids and lipids imitating the fed state (FeSSIF), slower in a buffer imitating the fasted state (FaSSIF), and slowest in a control buffer containing no bile components (FIG. 1A). Conversely, the flux rate, or permeation through an artificial lipid membrane, of asciminib was lowest in the fed state buffer (FeSSIF; 0.44 μg/minute; 94.2% dissolution), higher in the fasted state buffer (FaSSIF; 1.45 μg/minute; 93.1% dissolution), and highest in the control buffer (2.66 μg/minute; 82.3% dissolution) (FIG. 1B).

### Example 2: A Phase I, Single Center, Two-Group, Open-Label Study to Evaluate the Effects of Imatinib and Food on the Pharmacokinetics of ABL001 (Asciminib) in Healthy Volunteers

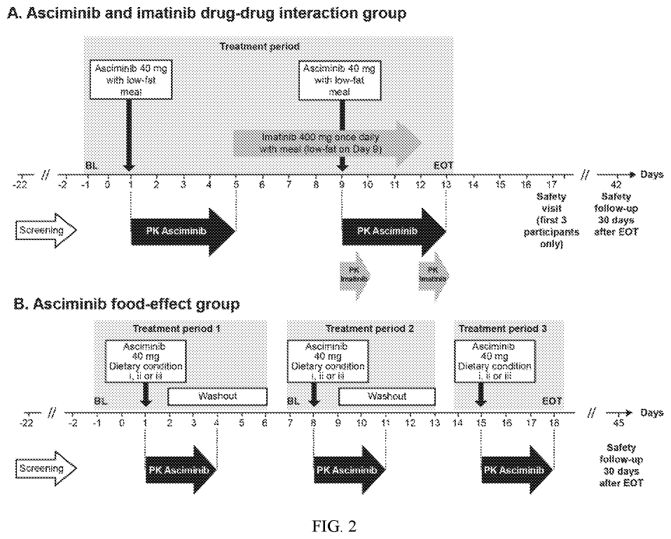

An open-label, single center phase I study, involving two separate groups of healthy volunteers—drug-drug interaction (DDI) study group and food effect (FE) study group. The two study groups were enrolled independently from each other. FIG. 2 is a schematic overview of the treatment protocol.

The primary objectives of this study are as follows:

### DDI Study Group

To evaluate the effect of multiple once-daily doses at steady-state of 400 mg imatinib on the pharmacokinetics of single dose of asciminib in healthy subjects under low-fat meal conditions.

### FE Study Group

To assess the FE on the oral bioavailability of a single dose of asciminib in healthy subjects under varying food conditions.

The endpoint (EP) for the primary objectives are as follows:
Primary pharmacokinetic parameters: Cmax, AUCinf, and AUClast of asciminib
Secondary pharmacokinetic parameters: Tmax, Tlast, AUC0-96h, Lambda_z, T1/2, CL/F, Vz/F of v.

The secondary objectives of the study are as follows:

### DDI Study Group

To evaluate the safety and tolerability of asciminib administered concomitantly with 400 mg imatinib under low-fat meal conditions in healthy subjects.
To evaluate the steady-state pharmacokinetics of 400 mg imatinib qd when administered in combination with a single dose of asciminib under low-fat meal conditions in healthy subjects.

### FE Study Group

To evaluate the safety and tolerability of single oral dose of asciminib administered in healthy subjects under varying food conditions.

The endpoint (EP) for the secondary objectives are as follows:
Safety parameters such as occurrence of adverse events and serious adverse events, changes in hematology and blood chemistry values, vital signs and electrocardiograms.
Secondary pharmacokinetic parameters: Tmax, Tlast, AUC0-96h, Lambda_z, T1/2, CL/F, Vz/F of imatinib in the DDI Study Group.

TABLE 1
Noncompartmental pharmacokinetic parameters of asciminib and imatinib

| | |
|---|---|
| Cmax | The maximum (peak) observed plasma drug concentration after single dose administration (ng × mL⁻¹) |
| Tmax | The time to reach maximum (peak) plasma drug concentration after single dose administration (hr) |
| AUClast | The AUC from time zero to the last measurable concentration sampling time (tlast) (ng × hr × mL⁻¹) |
| Tlast | Time of last measurable concentration (hr) |
| AUC0-24 h | The area under the plasma concentration-time curve (AUC) from time zero to 24 h post-dosing |
| | (Group 1-imatinib on Days 9 and 12) (ng × hr × mL⁻¹) |
| AUC0-96 h | The area under the plasma concentration-time curve (AUC) from time zero to 96 h post-dosing |
| | (Group 1-ABL001) (ng × hr × mL⁻¹) |
| AUC0-72 h | The area under the plasma concentration-time curve (AUC) from time zero to 72 h post-dosing |
| | (Group 2) (ng × hr × mL⁻¹) |
| AUCinf | The AUC from time zero to infinity (ng × hr × mL⁻¹) |
| Lambda_z | Smallest (slowest) disposition (hybrid) rate constant (time−1) may also be used for terminal |
| | elimination rate constant (hr⁻¹) |
| T1/2 | The elimination half-life associated with the terminal slope (λz) of a semi logarithmic |
| | concentration-time curve (hr) |
| CL/F | Apparent total body clearance of drug from plasma after oral administration (L/hour) |
| Vz/F | Apparent volume of distribution during terminal phase (associated with λz) after |
| | oral administration (L) |

### Study Design

This study is a phase I, single center, open-label, two-group design. Each subject underwent a screening period (Days −22 through −2), a pre-treatment period (baseline, Day −1), a treatment period, an end of treatment visit and a 30 days safety follow-up (phone call).

Study Group 1 (DDI Study Group) The DDI study group was a single sequence non-randomized group that evaluated the effect of multiple doses of 400 mg imatinib administered with a low-fat meal on the pharmacokinetics (PK) of asciminib. This group consisted of a screening period of up to 21 days, a baseline period (Day −1), and a treatment period of 13 days (Day 1 to Day 13) and an additional safety period of 30 days after the last dosing.

The first three subjects enrolled into DDI group were planned to undergo a safety phase of three additional days after the end of treatment visit. If no safety findings were observed in the first three subjects, the study was to continue to enroll the remaining 20 subjects. If safety findings meeting the criteria were observed in 1 of the 3 subjects, a further three subjects were planned to be enrolled and observed for a minimum period of 17 days prior to continuing further enrollment in the study.

The treatment sequence for each subject in Study Group 1 is shown in Table 1.
Day 1: A FDA low-fat meal was provided to the subjects after at least 10 hours of fasting. A single dose of 40 mg asciminib was administered 30 minutes±5 minutes after start of the meal.
Days 5-8: Imatinib 400 mg was administered once daily with a meal and a glass of water.
On Day 5, the blood sample for asciminib measurement was drawn before administration of imatinib.
Day 9: A FDA low-fat meal was provided to the subjects after at least 10 hours of fasting. A single dose of 400 mg imatinib and 40 mg asciminib was administered 30 minutes±5 minutes after start of the meal.
Days 10-12: Imatinib 400 mg was administered once daily with a meal and a glass of water.

TABLE 1
Study flow for Study Group 1

| | | | | | | | | | | | | | | | | |
|---|---|---|---|---|---|---|---|---|---|---|---|---|---|---|---|---|
| | | | | | | | | | | | | | | | D 17 | |
| | | | | | | | | | | | | | | | Safety | D 42 |
| | | | | | | | | | | | | | | D 13 | visit | (safety |
| D −22 to D −2 | D −1 | | | | | | | | | | | | | (end of | (3 first | follow-up |
| (screening) | (BSL) | D 1 | D 2 | D 3 | D 4 | D 5 | D 6 | D 7 | D 8 | D 9 | D 10 | D 11 | D 12 | treatment) | subjects) | call) |

| | | | | | | | | | |
|---|---|---|---|---|---|---|---|---|---|
| Asciminib dose | X* | | | | | X* | | | |
| Imatinib dose | | X | X | X | X | X | X | X | X |

| | | |
|---|---|---|
| PK sampling | PK (sampling) | PK (asciminib) |

| | | | | | | | | | |
|---|---|---|---|---|---|---|---|---|---|
| x | x | x | x | x | x | x | x | x | x |

| PK (imatinib) | | | |
|---|---|---|---|
| x | x | x | x |

*Dose administration with a low-fat meal.
BSL: baseline;
PK: Pharmacokinetics

### Study Group 2 (FE Study Group)

The FE study group was a cross-over and randomized group, that evaluated the effect of various food conditions on the PK of asciminib. The study consisted of a screening period of up to 21 days, three baseline periods (one before each treatment period) and three treatment periods (each separated by a 7-day washout) and an additional safety period of 30 days after the last dose. Each subject has undergone three treatment periods in which asciminib was administered either under fasting conditions, with a low-fat meal, or with a high-fat meal.

Subjects were randomized to one of six treatment sequences, and each treatment sequence had approximately four subjects. Each subject underwent three treatment periods separated by a 7-day washout starting from the dosing day of the previous treatment period until the baseline day of the next period (inclusive). The end-of-treatment (EOT) evaluation was conducted 3 days after administration of the last dose of study treatment. A safety follow-up phone call was placed 30 days after the last dosing.

The treatment sequence for each subject in Study Group 2 is shown in Table 2.

All subjects were to be administered a single oral dose of 40 mg asciminib tablet under various food conditions on Day 1, Day 8, and Day 15. All subjects received 3 doses of asciminib on Day 1, Day 8, and Day 15.

TABLE 2
Study flow for Study Group 2

| | Screening | | | | | | | | | |
|---|---|---|---|---|---|---|---|---|---|---|
| | | | | | | | | | | Safety |
| | Period 1 | | | Period 2 | | | Period 3 | | | follow-up |
| Day −22 | BL1 | Dose | Wash out | BL2 | Dose | Wash out | BL3 | Dose | EOT | phone call |
| to −2 | (Day −1) | (Day 1) | (Days 2-6) | (Day 7) | (Day 8) | (Days 9-13) | (Day 14) | (Day 15) | (Day 18) | Day 45 |

| | | | |
|---|---|---|---|
| Sequence 1 | A | B | C |
| Sequence 2 | B | C | A |
| Sequence 3 | C | A | B |
| Sequence 4 | C | B | A |
| Sequence 5 | A | C | B |
| Sequence 6 | B | A | C |

Abbreviations: BL1, BL2, BL3 = Baseline 1, 2, 3; EOT = End of Treatment evaluation: Pharmokinetics (PK) sampling during Days 1-4, Days 8-11, Days 15-18.
Treatment A: Single dose of 40 mg asciminib under fasting conditions.
Treatment B: Single dose of 40 mg asciminib with an FDA low-fat meal.
Treatment C: Single dose of 40 mg asciminib with an FDA high-fat meal.

### Dietary Requirements and Treatment Administration

During the screening process and throughout the study, the subjects were informed and reminded of restrictions to avoid strenuous physical exercise and sauna, and to avoid alcohol, foods containing poppy seeds, caffeinated food and beverages, and fruit (e.g., grapefruit, star fruit, cranberry, pummelos, pomegranate and Seville oranges) known to inhibit CYP3A4.

### Study Group 1 (DDI Study Group)

Asciminib was administered in the morning after an overnight fast of ≥10-hour fast, 30±5 minutes after the start of a FDA low-fat breakfast, together with 240 mL of water.
Imatinib was administered in the morning with a meal (standardized) and about 200 mL water.
On Day 9, imatinib and asciminib were administered 30±5 minutes after the start of a low-fat breakfast, together with 240 mL water; with imatinib taken first, immediately followed by asciminib.
No food was permitted for at least 4 hours after asciminib and asciminib+imatinib dosing, and at least 1 hour on days when imatinib was given alone.

### Study Group 2 (FE Study Group)

Asciminib was administered in the morning after an overnight fast of ≥10-hour with 240 mL of water, either under fasting conditions (i.e. no breakfast), 30±5 minutes after the start of a low-fat breakfast, or 30±5 minutes after the start of a high-fat breakfast.
For all 3 dietary conditions, participants were required to fast 4 hours after asciminib dosing.

The composition of high- and low-fat meals was based on guidance by the U.S. Food and Drug Administration (FDA), with high-fat meals comprising 800-1000 calories, ˜50% fat, ˜35% carbohydrates, and ˜15% protein, and low-fat meals comprising ≤400 calories and <20% fat (FDA Guidance on Food Effect Studies, December 2002).

Intake of fluids was not permitted from 1 hour pre-dose until 1 hour post-dose, except for fluid taken with breakfast before dosing, and water taken for study drug intake. Otherwise, water was taken as desired.

Participants were required to consume the entire contents of the meal provided within 30 minutes; any events of incomplete meal consumptions were recorded. Asciminib and imatinib were administered as film-coated tablets.

### Population

Approximately 47 healthy adult male and female subjects satisfying the inclusion and exclusion criteria were planned to be enrolled in the study.

### Main Inclusion Criteria

Adult male and/or female (sterile or postmenopausal) subjects 18-55 years of age, with a body mass index (BMI) of 18.0-29.9 kg/m², in good health condition as determined by no clinically significant findings from medical history, physical examination, vital signs and electrocardiogram (ECG), and laboratory tests were enrolled in the study.

### Main Exclusion Criteria

Cardiac or cardiac repolarization abnormalities, a history of immunodeficiency diseases, any surgical or medical conditions that could interfere with the absorption, distribution, metabolism, or excretion of study treatment, a history of malignancy of any organ system (other than localized basal cell carcinoma of the skin or in situ cervical cancer), and smoking.

### Pharmacokinetic Sampling and Assessments

In the DDI group, asciminib blood levels were assessed over Days 1-5 and Days 9-13, with samples collected pre-dose and at 0.5, 1, 2, 3, 4, 6, 8, 10 and 12 hours post-dose (Days 1 and 9), at 24 and 36 hours post-dose (Days 2 and 10), and at 48, 72 and 96 hours post-dose (Days 3-5 and Days 11-13).
Imatinib PK was assessed over Days 9-10, and Days 12-13, with samples collected pre-dose and at 0.5, 1, 2, 3, 4, 5, 6, 8, 10, and 12 hours post-dose (Days 9 and 12), and at 24 hours post-dose (Days 10 and 13).
In the FE group, asciminib PK was assessed over Days 1-4 of each of the 3 treatment periods, with samples collected pre-dose and at 0.5, 1, 2, 3, 4, 6, 8, 10, and 12 hours post-dose (Day 1), at 24 and 36 hours post-dose (Day 2), and at 48 and 72 and 96 hours post-dose (Days 3-4).
Plasma concentrations of asciminib and imatinib were determined using a validated liquid chromatography-tandem mass spectrometry assay (LC-MS/MS) with a dynamic range of 1.00-5000 ng/mL for asciminib, and 20.0-10,000 ng/mL for imatinib. The method was validated for specificity, sensitivity, matrix effect, recovery, linearity, accuracy and precision, dilution integrity, batch size and stability.
For the analysis of asciminib, the accuracy and precision for the LLOQ (1.00 ng/mL) were within ±5.0% bias and ≤8.0% CV, respectively. Based on intra-day and inter-day evaluations, the accuracy (% bias) of the other internal standard solution samples ranged from −2.0 to 5.3%, and the precision from 2.8 to 6.2% CV.
For the analysis of imatinib, the accuracy and precision for the LLOQ (20.0 ng/ml) were within ±1.5% bias and ≤8.8% CV, respectively. Based on intra-day and inter-day evaluations, the accuracy (% bias) of the other internal standard solution samples ranged from −5.0% to 1.8%, and the precision from 3.4% to 7.4% CV.

### Statistical Analyses

Assuming an intra-subject variability of 30% for the primary asciminib PK parameters (based on previously reported data 17), a sample size of 18 participants per group was estimated to provide adequate precision of the 90% CIs for the difference between test and reference parameters on the log scale, with an error margin for the observed difference in means of 0.170 in the DDI group, and 0.166 in the FE group (based on a paired t-test with 2-sided alpha level of 0.10). Considering a potential dropout rate of 20%, 23 participants were to be enrolled into the DDI group, and 24 participants in the FE group.
PK analyses were based on all participants with at least 1 evaluable PK profile.
In the DDI group, a participant's PK profile was considered evaluable if they had received all planned doses of imatinib on Days 5-9 (Day 9 profile); all planned doses of imatinib on Days 5-9 and at least 2 of the planned imatinib doses on Days 10-12 (Day 12 profile); received the planned doses of asciminib on the respective day; fulfilled the pre-specified fasting requirements; had not vomited within 4 hours after the dosing of asciminib and/or imatinib, and provided at least 1 primary PK parameter for asciminib (asciminib PK profile) or imatinib (imatinib PK profile).
In the FE group, a participant's asciminib PK profile was considered evaluable if they had received 1 of the planned treatments; had consumed at least 75% of the meal for the respective fed treatments; fulfilled the pre-specified treatment administration requirements; provided at least 1 primary asciminib PK parameter; fulfilled the pre-specified fasting requirements; and had not vomited within 4 hours after the dosing of asciminib.
The safety sets in both groups comprised all participants who received ≥1 dose of study treatment. PK parameters were calculated from individual plasma concentration-time profile using non-compartmental methods using Phoenix WinNonlin® (Pharsight, Mountain View, CA) software version 6.4. PK parameters were summarized using the geometric mean (Gmean), geometric coefficients of variation (GCV %), median, minimum, and maximum. Baseline characteristics are presented as frequencies and percentages for categorical data, and as median, minimum, and maximum for continuous data. Missing values, and values below the LLOQ, were treated as missing in calculations of Gmean and GCV %. Formal statistical comparisons were performed for the primary asciminib PK parameters of Cmax, AUCinf, and AUClast. In the DDI group, the formal statistical comparison assessed asciminib+imatinib (test) vs asciminib alone (reference); a linear mixed model was fitted to the log-transformed PK parameters, with treatment included as a fixed factor and participant as random factor. In the FE group, formal statistical comparisons assessed low-fat meal (test) vs fasting (reference), and high-fat meal (test) vs fasting (reference); a linear mixed model was fitted to the log-transformed PK parameters, with treatment, period and sequence included as fixed factors and participant nested within sequence as random factor. For all comparisons, the point estimate and corresponding 2-sided 90% confidence interval (CI) for the difference between test and reference were calculated. The point estimate and CI were anti-log transformed to obtain the point estimate and the 90% CI for the geometric mean (Gmean) ratio on the original scale. The characterization of secondary PK parameters was descriptive only. All analyses were performed using Statistical Analysis System (SAS) version 9.4.

### Safety

Monitored by assessing physical examination, vital signs, height and weight, laboratory evaluations, cardiac assessments, meal records as well as collection of the adverse events (AEs) at every visit.
   AEs were coded using Medical Dictionary for Regulatory Activities (MedDRA) Version 20.1, and the Common Terminology criteria for AEs [CTCAE] Version 4.03.
All clinical samples analysis (hematology, blood chemistry) was performed by the local laboratory.

### Results

### Participant Disposition and Baseline Characteristics

Overall, 47 participants were enrolled in the study of whom 23 participants were enrolled in the DDI group and 24 participants in the FE group.
In the DDI group, the first 3 enrolled participants underwent a safety phase of 3 additional days after the end of treatment visit (sentinel dosing). No safety findings were observed in these 3 participants, and thus the remaining 20 participants were enrolled. The DDI study was completed by 22 participants, with all 22 participants receiving all planned doses of asciminib on Days 1 and 9, and all 8 doses of imatinib from Day 5 to 12. One participant discontinued the study on Day 5 and was excluded from the imatinib PK analysis set per protocol due to vomiting within 4 hours of imatinib dosing (Grade 1 vomiting).
In the FE group, all 24 enrolled participants (4 participants in each of the 6 sequences) completed the study, with all participants receiving all planned doses of asciminib. One participant did not receive asciminib within 30±5 minutes after start of the meal during one of the treatment periods and was excluded from the PK analysis.
In the DDI group, median age at baseline was 47.0 years (range: 23-55 years), and median body mass index (BMI) was 25.3 (range 19.1-29.5); 87.0% (20/23) participants were male, and 95.7% (22/23) were White, with 1 participant being African American.
In the FE group, median (range) age was 44.5 (21-54) years, median BMI was 24.65 (range 19.7-29.4), 95.8% (23/24) of participants were male, and all participants were White.
One participant in the DDI group received concomitant paracetamol for headache (500 mg QD) on Days 5 and 6. No concomitant medication was administered in the FE group.

### PK Analyses DDI Group

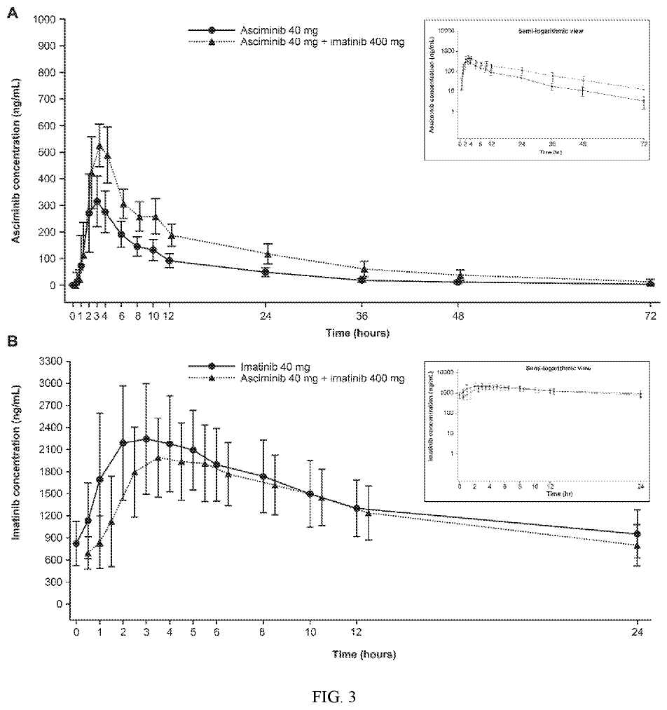

The plasma concentration-time profiles of asciminib revealed higher exposure when asciminib was administered together with imatinib at steady-state, compared with when asciminib was administered alone (FIG. 3A).
For both regimens, a rapid absorption followed by a biphasic decline after attaining Cmax was observed.
Descriptive PK parameters showed higher exposure to asciminib when administered in combination with imatinib than with asciminib monotherapy (Table 1). Median Tmax was similar regardless of whether asciminib was administered alone (3.00 h; range 1.01-6.00 hours), or together with imatinib (3.00 h; range 1.94-4.00 hours). The Gmean of T1/2 was comparable between asciminib with (15.3 hours) and without imatinib (13.7 hours). The Gmean of asciminib clearance was 5.19 L/h when asciminib was administered with imatinib, but higher at 11.1 L/h when asciminib was administered alone.
A statistical comparison of the primary asciminib PK parameters showed that with asciminib+imatinib, systemic asciminib exposure increased approximately 2-fold compared with asciminib alone (Gmean ratio [90% CI] asciminib+imatinib vs asciminib, AUCinf 2.08 [1.93; 2.24]; AUClast 2.07 [1.92; 2.23]), and Cmax 1.6 (Gmean ratio [90% CI] 1.59 [1.45; 1.75]) (Table 3).
Inter-subject variabilities (GCV %) for AUCinf and AUClast were comparable between asciminib alone and asciminib+imatinib (31.7-31.8% vs 27.0-28.0) and were higher for Cmax with asciminib alone than with asciminib+imatinib (33.4% vs 16.1%) (Table 4).

The plasma concentration-time profile of imatinib showed that when asciminib and imatinib were co-administered, imatinib exposure was slightly lower and slightly delayed compared with imatinib administered alone.
This effect was mainly observed during the absorption phase, with a delayed imatinib absorption in the presence of asciminib (FIG. 3B).
Imatinib PK parameters with asciminib+imatinib vs imatinib alone were Gmean AUClast 29,600 ng×h/mL (GCV % 27.3%) vs 33,600 ng×h/mL (GCV % 28.6%), respectively, Gmean AUC0-24h 30,100 ng x/mL (GCV % 27.4%) vs 33,600 ng×h/mL (GCV % 28.6%), respectively, and Gmean Cmax 2020 ng/mL (GCV % 26.9%) vs 2340 ng/mL (GCV % 29.5%), respectively. Median Tmax of imatinib was similar whether imatinib was administered with asciminib (3.01 [range 1.95-5.00] hours) or alone (3.00 [range 0.993-5.95] hours).

TABLE 3
Statistical comparison of asciminib PK parameters with
asciminib + imatinib vs asciminib alone (DDI group)

| | | | | Treatment comparison | | |
|---|---|---|---|---|---|---|
| PK | | | Adjusted | | Gₘₑₐₙ | 90% |
| parameter | Treatment | n | Gₘₑₐₙ | Comparison | ratio | CI |
| AUC_{inf} | asciminib | 23 | 3600 | | 2.08 | 1.93; 2.24 |
| (ng × h/mL) | asciminib + | 22 | 7490 | asciminib + | | |
| | imatinib | | | imatinib/asciminib | | |
| AUCₗₐₛₜ | asciminib | 23 | 3560 | asciminib + | 2.07 | 1.92; 2.23 |
| (ng × h/mL) | asciminib + | 22 | 7370 | imatinib/asciminib | | |
| | imatinib | | | | | |
| Cₘₐₓ | asciminib | 23 | 329 | asciminib + | 1.59 | 1.45; 1.75 |
| (ng/mL) | asciminib + | 22 | 525 | imatinib/asciminib | | |
| | imatinib | | | | | |

TABLE 4
Asciminib PK parameters with asciminib alone
and asciminib + imatinib (DDI group)

| | | Asciminib | Asciminib 40 mg + |
|---|---|---|---|
| | | 40 mg | imatinib 400 mg |
| Parameter | Statistics | (n = 23) | (n = 22) |
| AUC_{inf} | Gₘₑₐₙ | 3600 | 7710 |
| (ng × h/mL) | GCV % | 31.7 | 28.0 |
| | Median | 3750 | 7540 |
| | Range | 1590-7160 | 5280-12800 |
| AUCₗₐₛₜ | Gₘₑₐₙ | 3560 | 7580 |
| (ng × h/mL) | GCV % | 31.8 | 27.0 |
| | Median | 3720 | 7450 |
| | Range | 1570-7070 | 5210-12200 |
| Cₘₐₓ | Gₘₑₐₙ | 329 | 537 |
| (ng/mL) | GCV % | 33.4 | 16.1 |
| | Median | 349 | 529 |
| | Range | 134-514 | 391-757 |
| Tₘₐₓ | Gₘₑₐₙ | NA | NA |
| (h) | GCV % | NA | NA |
| | Median | 3.00 | 3.00 |
| | Range | 1.01-6.00 | 1.94-4.00 |
| T_{1/2} | Gₘₑₐₙ | 13.7 | 15.3 |
| (h) | GCV % | 16.8 | 20.2 |
| | Median | 13.9 | 14.5 |
| | Range | 10.6-19.0 | 11.3-23.2 |

### PK Analyses FE Group

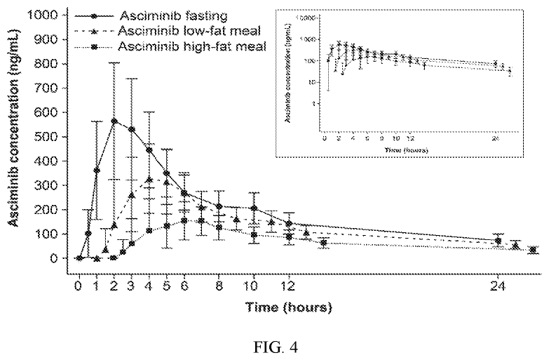

The plasma concentration-time profiles of asciminib under different food conditions indicated that compared with fasting conditions, administration together with a meal decreased asciminib exposure and delayed Tmax, particularly with a high-fat meal (FIG. 4).
Likewise, descriptive PK parameters showed lower exposure to asciminib when administered with a high- or low-fat meal compared with fasting conditions, with a greater decrease in exposure observed with high-fat than with low-fat meals (Table 5).
Median Tmax (range) of asciminib was longer when asciminib was administered with a high-fat meal (4.01 [1.00-8.00] hours) or low-fat meal (3.00 [0.997-5.00] hours), compared with the fasted state (2.01 [1.00-5.00] hours).

These observations were confirmed in a statistical comparison of asciminib PK parameters, which showed that the higher the fat content of the accompanying meal, the lower the exposure to asciminib (Table 6).
For example, the Gmean ratio for AUCinf was 0.700 (90% CI 0.631-0.776) under low-fat meal conditions, indicating a 30% reduction in exposure compared with fasted conditions.
Under high-fat meal conditions, the Gmean ratio for AUCinf was 0.377 (90% CI 0.341-0.417), indicating a 62.3% reduction in exposure compared with the fasted state. A similar pattern was observed for AUClast and Cmax. Inter-subject variability for the primary PK parameters were slightly higher when asciminib was administered with a high-fat meal (GCV % AUCinf 43.9%; AUClast 43.8%; Cmax 51.3%) compared with a low-fat meal (GCV % AUCinf 29.1%; AUClast 29.0%; Cmax 39.8%), or fasting conditions (GCV % AUCinf 35.5%; AUClast 35.2%; Cmax 39.7%) (Table 5).

The observed food-effect on the PK of asciminib in healthy individuals was also supported by an experimental in vitro flux study in Example 1, which assessed the impact of different compositions of bile on how a 40 mg dose of asciminib dissolves and permeates through an artificial lipid membrane.

TABLE 5
Asciminib PK parameters with asciminib administered under fasted
conditions, or after a low-fat or high-fat meal (FE group)

| | | Asciminib | Asciminib | Asciminib |
|---|---|---|---|---|
| | | 40 mg | 40 mg | 40 mg |
| | | Fasted | Low-fat meal | high-fat meal |
| Parameter | Statistics | (n = 24) | (n = 23) | (n = 24) |
| AUC_{inf} | Gₘₑₐₙ | 5830 | 4130 | 2200 |
| (ng × h/mL) | GCV % | 35.5 | 29.1 | 43.9 |
| | Median | 5890 | 4300 | 2040 |
| | Range | 2820-10400 | 2620-6960 | 867-4330 |
| AUCₗₐₛₜ | Gₘₑₐₙ | 5730 | 4060 | 2130 |
| (ng × h/mL) | GCV % | 35.2 | 29.0 | 43.8 |
| | Median | 5820 | 4270 | 1960 |
| | Range | 2810-10200 | 2560-6840 | 845-4210 |
| Cₘₐₓ | Gₘₑₐₙ | 550 | 363 | 175 |
| (ng/mL) | GCV % | 39.7 | 39.8 | 51.3 |
| | Median | 572 | 365 | 159 |
| | Range | 280-1050 | 172-709 | 82.2-423 |
| Tₘₐₓ | Gₘₑₐₙ | NA | NA | NA |
| (h) | GCV % | NA | NA | NA |
| | Median | 2.01 | 3.00 | 4.01 |
| | Range | 1.00-5.00 | 0.997-5.00 | 1.00-8.00 |
| T_{1/2} | Gₘₑₐₙ | 13.5 | 13.5 | 12.8 |
| (h) | GCV % | 22.1 | 16.7 | 23.0 |
| | Median | 14.5 | 14.0 | 12.9 |
| | Range | 9.18-19.1 | 9.70-17.7 | 8.81-18.1 |

TABLE 6
Statistical comparison of asciminib PK parameters with asciminib administered
under fasted conditions, or after a low-fat or high-fat meal (FE group)

| | | | | Treatment comparison | | |
|---|---|---|---|---|---|---|
| PK | | | Adjusted | | Gₘₑₐₙ | 90% |
| parameter | Treatment | n | Gₘₑₐₙ | Comparison(s) | ratio | CI |
| AUC_{inf} | asciminib fasted | 24 | 5830 | asciminib + low-fat | 0.700 | 0.631; 0.776 |
| (ng × h/mL) | asciminib + | 23 | 4080 | meal/asciminib fasted | | |
| | low-fat meal | | | | | |
| | asciminib + | 24 | 2200 | asciminib + high-fat | 0.377 | 0.341; 0.417 |
| | high-fat meal | | | meal/asciminib fasted | | |
| AUCₗₐₛₜ | asciminib fasted | 24 | 5730 | Asciminib + low-fat | 0.700 | 0.630; 0.777 |
| (ng × h/mL) | asciminib + | 23 | 4010 | meal/asciminib fasted | | |
| | low-fat meal | | | | | |
| | asciminib + | 24 | 2130 | Asciminib + high-fat | 0.372 | 0.336; 0.413 |
| | high-fat meal | | | meal/asciminib fasted | | |
| Cₘₐₓ | asciminib fasted | 24 | 550 | asciminib + low-fat | 0.652 | 0.576; 0.739 |
| (ng/mL) | asciminib + | 23 | 359 | meal/asciminib fasted | | |
| | low-fat meal | | | | | |
| | asciminib + | 24 | 175 | asciminib + high-fat | 0.318 | 0.282; 0.360 |
| | high-fat meal | | | meal/asciminib fasted | | |

### Safety

In both study groups, single doses of 40 mg asciminib were well tolerated.
This was also the case for asciminib+imatinib sentinel dosing cohort in the DDI group, and thus enrollment and treatment was extended to the full DDI cohort.
In the DDI group, at least 1 AE was reported in 14 participants (60.9%). The most frequently reported AEs (≥5% of participants) were headache (n=5, 21.7%) and arthralgia, erythema, fatigue, feeling hot, nasal congestion, nasopharyngitis, and vomiting (each n=2, 8.7%).
In the FE group, 11 participants (45.8%) had at least 1 AE; the most frequently reported AEs were oropharyngeal pain and rhinitis (each n=2, 8.3%).
In both study groups, all AEs were CTCAE Grade 1 or 2, and there were no serious AEs. There were no clinically significant abnormalities in laboratory evaluations, vital signs, or ECG.
In the DDI group, 1 participant had Grade 3 decreased neutrophils, and Grade 2 decreased leukocytes.
In the FE group, 1 participant had Grade 3 elevated triacylglycerol lipase on Day 2 of the fasted treatment period which improved to normal on Day 3. Another participant in the FE group had Grade 2 elevated triglycerides on Days 2-6 of the low-fat treatment period and on Day 9 of the fasting period until EOT, Grade 2 increased cholesterol on Day 14 of the high-fat treatment period until EOT which returned to normal on Day 31, and Grade 3 increased lipase on Day 16 of the high-fat treatment period which resolved to normal on Day 18. There were no other hematological or biochemical abnormalities of Grade 3 or higher in either the DDI or the FE groups.

### DISCUSSION

This Phase 1 study assessed the impact of imatinib steady-state (under low-fat meal conditions) or varying food conditions on the PK of a single dose of asciminib 40 mg (FMI tablet formulation) in healthy volunteers.
Imatinib, and therefore the combination of asciminib+imatinib, are administered with a meal to minimize gastric discomfort.
The DDI study compared asciminib with and without imatinib when taken under the same standard FDA low-fat meal conditions and hence, the difference in PK between the two situations is assumed to reflect the DDI between asciminib and imatinib.
Asciminib+imatinib (taken with a low-fat meal) resulted in a 2-fold increase in asciminib systemic exposure (AUCinf and AUClast), and a 1.6-fold increase in asciminib Cmax, compared with single-agent asciminib (taken under the same food conditions).
Asciminib Tmax did not differ substantially between asciminib alone and asciminib+imatinib. PK parameters of imatinib observed in the present study were comparable to those previously reported in an imatinib single-agent dose-finding study in patients with CML (Peng et. al., Clinical pharmacokinetics of imatinib. Clin Pharmacokinet 2005; 44(9): 879-94), and also to those observed with imatinib+asciminib in patients with CML (Cortes et. al. Combination Therapy Using Asciminib Plus Imatinib in Patients With Chronic Myeloid Leukemia: Results From a Phase 1 Study. Presented at the European Hematology Association 24th Annual Congress; 2019; Amsterdam, the Netherlands) indicating that the potential for asciminib to affect the PK of imatinib is low.

The observed effects of imatinib on asciminib exposure can be attributed to the fact that imatinib inhibits multiple pathways that are involved in the metabolism of asciminib.
Asciminib undergoes direct glucuronidation via several UDP-glucuronosyltransferases (UGTs; mainly UGT2B7 and UGT2B17), and oxidation predominantly via CYP3A4, and is also a substrate of breast cancer resistance protein (BCRP) and biliary secretion contributes to its clearance (Tran et al., Disposition of asciminib, a potent BCR-ABL1 tyrosine kinase inhibitor, in healthy male subjects. Xenobiotica 2020; 50(2): 150-69).
Imatinib, on the other hand, is an in vitro reversible moderate inhibitor of CYP3A4,14 an inhibitor of various UGTs with high potency for UGT2B17,23 and an inhibitor of BCRP and P-glycoprotein (D'Cunha et al., TKI combination therapy: strategy to enhance dasatinib uptake by inhibiting Pgp- and BCRP-mediated efflux. Biopharm Drug Dispos 2016; 37(7): 397-408).

Importantly, the 2-fold increase in asciminib exposure observed in the study with asciminib+imatinib is not expected to have a negative effect on the safety profile of the combination regimen, as in patients with CML, the maximum tolerated dose of single-agent asciminib was not reached with doses of up to 200 mg twice daily (Hughes et al., Asciminib in Chronic Myeloid Leukemia after ABL Kinase Inhibitor Failure. N Engl J Med 2019; 381(24): 2315-26).
Indeed, AE data from the study, including those from the sentinel dosing cohort, together with available safety data of the combination in patients with CML, demonstrate that asciminib+imatinib was well tolerated, with a safety profile consistent with that of single-agent asciminib9 and with no new safety signals.

Results from the FE analysis demonstrated a decrease in asciminib exposure when asciminib was administered with food. This effect was more pronounced with a higher fat content of the meal, with asciminib AUCinf and AUClast decreasing by 30% with a low-fat meal, and by 62-63% with a high-fat meal, compared with fasted conditions. A delay in Tmax was also observed when asciminib was administered with food compared with fasting conditions, and as before for exposure, the shift in Tmax increased with meal fat content. Possibly, the observed FE of asciminib is related to its sequestration with bile acids, in particular when the concentration of bile acids in the gastrointestinal tract is high, such as after a high-fat meal. This notion is supported by the results of the flux analysis, which showed that the higher the concentrations of bile components in the buffer (i.e. the higher the fat content of the consumed meal 20), the lower the flux rate of asciminib (i.e. the longer Tmax and the lower the absorbed fraction of asciminib in vivo).

The magnitude of the FE, as well as the absolute measures of asciminib AUCinf, AUClast and Cmax observed with the asciminib FMI tablet formulation in the present study, are in line with previous results on the FE using initial asciminib formulations. With these initial asciminib tablet formulations, asciminib exposure decreased independent of the tablet variant by ˜30% and ˜65% with a low-fat and high-fat meal, respectively, compared with fasted state.15 Based on these FE findings, asciminib (FMI tablet formulation) as single-agent is to be administered in the fasted state.

### CONCLUSION

The study shows that co-administration of asciminib (FMI tablet formulation) with imatinib results in a moderate (2-fold) increase in asciminib exposure compared with asciminib alone when taken under the same food conditions, with no change in imatinib exposure. Hence, when used in combination with imatinib (with food), asciminib is dosed at 40 mg or 60 mg once daily compared with the recommended asciminib single-agent dose of 40 mg twice daily in the fasted state (Saglio et al., Randomized, Open-Label, Multicenter, Phase 2 Study of Asciminib (ABL001) As an Add-on to Imatinib Versus Continued Imatinib Versus Switch to Nilotinib in Patients with Chronic Myeloid Leukemia in Chronic Phase Who Have Not Achieved a Deep Molecular Response with Frontline Imatinib. ASH; 2019; 2019. p. (Supplement 1): 5910). Food itself, in particular high-fat food, leads to a moderate (30-60%) decrease in asciminib exposure, depending on fat content, which is possibly related to the sequestration of asciminib with bile acids. Therefore, to avoid suboptimal exposure, asciminib as a single-agent is recommended to be administered in the fasted state. The combination of asciminib+imatinib was well tolerated in this study in healthy volunteers, which is in line with the preliminary clinical experience in patients with CML. Overall, the findings indicate that co-administration of imatinib 400 mg once daily with asciminib 40 mg once daily under low-fat meal conditions resulted in a moderate increase of asciminib exposure, similar to that provided with the recommended asciminib monotherapy dose (40 mg twice daily) under fasting conditions, with a favorable safety profile. Asciminib 40 mg or 60 mg once daily are currently under further investigation as an add-on therapy to imatinib in a Phase 2, randomized trial in patients with CML in chronic phase who have received first-line imatinib and have not achieved a deep molecular response (NCT03578367).

All publications and patent documents cited herein are incorporated herein by reference as if each such publication or document was specifically and individually indicated to be incorporated herein by reference. The present invention and its embodiments have been described in detail. However, the scope of the present invention is not intended to be limited to the particular embodiments of any process, manufacture, composition of matter, compounds, means, methods, and/or steps described in the specification. Various modifications, substitutions, and variations can be made to the disclosed material without departing from the spirit and/or essential characteristics of the present invention. Accordingly, one of ordinary skill in the art will readily appreciate from the invention that later modifications, substitutions, and/or variations performing substantially the same function or achieving substantially the same result as embodiments described herein may be utilized according to such related embodiments of the present invention. Thus, the following claims are intended to encompass within their scope modifications, substitutions, and variations to processes, manufactures, compositions of matter, compounds, means, methods, and/or steps disclosed herein. The claims should not be read as limited to the described order or elements unless stated to that effect. It should be understood that various changes in form and detail may be made without departing from the scope of the appended claims.

1. A method of treating a BCR-ABL mediated disease or disorder in a patient in need thereof, comprising administering a pharmaceutical combination comprising (i) a therapeutically effective amount of asciminib or a pharmaceutically acceptable salt thereof and (ii) a therapeutically effective amount of at least one further therapeutic agent; wherein the pharmaceutical combination is administered together with food.

2. The method of claim 1, wherein the BCR-ABL mediated disease or disorder is a leukemia selected from chronic myeloid leukemia (CML), acute lymphoblastic leukemia (ALL), and acute myeloid leukemia (AML).

3. The method according to claim 1, wherein asciminib or a pharmaceutically acceptable salt thereof is used in add-on combination therapy to the one further therapeutic agent.

4. The method according to claim 1, wherein asciminib or a pharmaceutically acceptable salt thereof is administered to the patient at a total daily dose of about 40 mg or 60 mg in a single dose.

5. The method according to claim 1, wherein the one further therapeutic agent is selected from imatinib, nilotinib, dasatinib, bosutinib, ponatinib and bafetinib.

6. The method of claim 5, wherein the one further therapeutic agent is imatinib.

7. The method according to claim 1, wherein imatinib is administered to the patient at a total daily dose of about 400 mg in a single dose.

8. The method according to claim 1, wherein the food is a low-fat meal.

9. The method according to claim 1, wherein the pharmaceutical combination is administered together sequentially or simultaneously.

10. A method of treating a BCR-ABL mediated disease or disorder in a patient in need thereof, comprising administering a total daily dose of about 40 mg or 60 mg of asciminib, or a pharmaceutically acceptable salt thereof, in a single dose and a total daily dose of about 400 mg of imatinib in a single dose, wherein the single dose of asciminib, or a pharmaceutically acceptable salt thereof, and the single dose of imatinib are administered together with a low-fat meal.

11. The method according to claim 10, wherein the dose of asciminib or a pharmaceutically acceptable salt thereof and the dose of imatinib are administered simultaneously.

12. The method according to claim 11, wherein asciminib or a pharmaceutically acceptable salt thereof is administered in add-on combination therapy.

13. A method of treating a BCR-ABL mediated disease or disorder in a patient in need thereof, comprising administering a therapeutically effective amount of asciminib or a pharmaceutically acceptable salt thereof without food.

14. The method according to claim 13, wherein the administration of asciminib or a pharmaceutically acceptable salt thereof with food results in a decrease in bioavailability in the subject as compared to the administration of asciminib or a pharmaceutically acceptable salt thereof without food.

15. The method according to claim 13, wherein the BCR-ABL mediated disease or disorder is a leukemia selected from chronic myeloid leukemia (CML), acute lymphoblastic leukemia (ALL), and acute myeloid leukemia (AML).

16. The method according to claim 13, wherein asciminib or a pharmaceutically acceptable salt thereof is administered to the patient at a total daily dose of about 80 mg in divided doses.
